(19) United States
(12) Patent Application Publication
(10) Pub. No.: US 2011/0086363 A1
(43) Pub. Date: Apr. 14, 2011

(54) Method and apparatus to conduct kinetic analysis of platelet function in whole blood samples

(76) Inventors: Bulent Mutus, Windsor (CA); Edward C.J. Carriveau, Windsor (CA); Harmanpreet Kaur, Windsor (CA)

(21) Appl. No.: 12/923,729

(22) Filed: Oct. 5, 2010

Related U.S. Application Data
(60) Provisional application No. 61/272,553, filed on Oct. 6, 2009.

(51) Int. Cl. G01N 33/53; A61L 33/00; C12M 1/34

(52) U.S. Cl. 435/7.1; 427/2.12; 435/287.9

(57) ABSTRACT

Adhesion of platelets to blood vessel walls is the first step that promotes arrest of bleeding by interaction of the platelet receptors with various extracellular matrix proteins that become exposed on vascular injury. A flow chamber is provided for use in analyzing or studying platelet function, in whole blood, either as part of a batch process or in real time. In the flow chambers, an inert polydimethylsiloxane (PDMS) surface is plasma-activated and a homobifunctional cross-linker is used to immobilize platelet-binding proteins onto a chamber wall surface. Immobilized collagen and fibrinogen may thus be assayed by continuously monitoring the adhesion of ADP and Ca²⁺ activated platelets from a subject, such as a patient having normal or type 2 diabetes (T2D). The flow chamber provides a simplified and robust method for the construction flow chambers which enable the kinetic monitoring of platelet adhesion in whole blood.

### RELATED APPLICATIONS

This application claims the benefit of 35 USC §119(e) to U.S. Provisional Application Ser. No. 61/272,553, filed Oct. 6, 2009.

### SCOPE OF THE INVENTION

The present invention relates generally to an apparatus for performing kinetic analysis of platelet binding and/or aggregation and which includes a sample flow chamber configured to receive a blood sample therein. More preferably, the flow chamber includes one or more surfaces having thereon a polymer base cross-linked to homobifunctional cross-linkers used to selectively bind and/or aggregate platelet binding proteins and/or blood platelets thereon.

### BACKGROUND OF THE INVENTION

Platelet adhesion and aggregation is the first step in the physiological defense mechanism of the body in blood vessel injury. Exposure of extracellular matrix at the sites of subendothelial disruption leads to the capture of platelets to the matrix through various receptors including GPIb-IX-V and GPVI, resulting in platelet activation, followed by thrombi formation.

Earlier investigations on platelet function have focused on the platelet adhesion to fibrinogen or collagen under flow conditions. In all of these studies, glass slides are coated with fibrinogen, collagen or von Willebrand factor and then used to study platelet aggregation mechanisms. Whilst flow cells having parallel plate flow chambers which mimic the in vivo conditions are known for platelet aggregation studies under various physiological conditions, typically conventional flow cells and microfluidic devices are expensive or lack reliability.

### SUMMARY OF THE INVENTION

There is currently little to no availability of simple, reliable, robust and inexpensive flow chambers for use in kinetic aggregation studies. Accordingly, one object of the present invention is to provide for the construction and testing of a simplified flow chamber, which fits onto an inverted microscope, and which permits the simplified optical monitoring and/or assay of the binding of fluorescently-labelled platelets, in whole blood, onto platelet-binding proteins covalently attached to a small area on the flow chamber's surface under in vivo-like conditions. More preferably, the flow cell has been validated by comparing kinetics of adhesion of resting as well as Ca²⁺ and ADP activated platelets from normal and Type 2 diabetic subjects onto fibrinogen or collagen covalently attached to the flow chamber's surface.

Another object of the invention is to provide an improved apparatus to overcome the disadvantages of the commercially available flow cells, and which provides simpler, inexpensive and more reliable flow chambers operable to allow kinetic analysis of a component in a blood sample, as for example may be used to diagnose blood or circulatory disorders.

In one possible construction, a flow cell is provided for use in analysing the adhesion and/or aggregation performing kinetic analysis of platelets in a blood sample comprises a sample flow chamber and non-aligned blood inflow and blood outlet channels providing fluid communication into and from the flow chamber. At least one wall position of the chamber supports a bed of collagen or fibrinogen proteins. More preferably a layer of polydimethylsiloxane is covalently cross-linked to collagen or fibrinogen by way of homodifunctional cross-linkers for binding and aggregating platelets in a whole blood sample. An optical or electron microscope may be coupled to a sidewall of the flow chamber for carrying our kinetic analysis.

In another aspect of the present invention, there is provided an apparatus for use in performing kinetic analysis of platelet binding and aggregation to fibrinogen or collagen in a blood sample. The apparatus includes a parallel plate flow chamber in fluid communication between a blood inlet passage and a blood outlet passage. The parallel plate flow chamber is defined by a plurality of sidewalls and at least one optically transparent end wall, which for example may be optically coupled to an optical or electron microscope. At least one end wall which is coated with hydrophobic polymer which is cross-linked with cross-linkers to fibrinogen or collagen.

In one possible use, a blood sample is prepared by isolating and fluorescent labelling the platelets therein and the platelets are placed back in a whole blood sample. The whole blood sample is perfused through the parallel plate flow chamber at a constant rate immediately after platelet activation with ADP or Ca²⁺. Fluorescent data corresponding to fluorescent labelled platelets bound to the fibrinogen or collagen are thereafter gathered by the inverted optical microscope at timed intervals.

More preferably, the applicant has appreciated that polydimethylsiloxane (PDMS) may be used advantageously to enable more reliable immobilization of proteins on the optically transparent flow chamber walls and particularly chamber wall surfaces made of glass. Furthermore, PDMS has been found in the art to have high affinity for proteins due to its hydrophobic nature. Hydrophobic proteins such as collagen and fibrinogen may therefore be more easily covalently linked to PDMS by means of homobifunctional cross-linkers.

In one simplified construction the flow chamber is closed at least one end by an optically transparent glass cover. The interior surfaces of the chamber is provided with an inert polydimethylsiloxane (PDMS) that is plasma oxidized using a plasma cleaner and a homobifunctional cross-linker was used to immobilize platelet binding proteins onto a small area of the surface at the centre of the flow chamber. This may be done by adding 0.2 μl of 2% aminopropyltrimethoxysilane (APTMES) to a roughly 1 mm square surface in the axial centre of the flow chamber. After 10 minutes a 0.5 μl portion of 0.5 mM disuccinimidyl substrate (DSS) solution was added to the APTMES dot at the geometric centre of the flow chamber. After 5 seconds a type 1 fibrinogen from bovine plasma solution with a final concentration of 5 μM was added. Alternately for analysis with collagen immobilization, a solution of 8 μM of collagen type 1 from rat tail is used. After 15 minutes the reaction is stopped by adding Tris buffer pH 8.

The prepared flow chamber may advantageously be fitted to an inverted optical microscope to permit simplified optical monitoring during perfusion studies.

As a result of using PDMS as a highly reliable protein immobilization medium, the present invention provides a suitable apparatus for applications requiring high degrees of precision such as diagnosing blood or circulatory disorders with known deviations in the kinetics of platelet aggregation, including, but not limited to, Type II diabetes, hemophilia, coronary artery disease, percutaneous coronary intervention and coronary stent implantations. This may be achieved in one aspect by using the apparatus for kinetic analysis of a patient's blood sample and comparing the data to those of a healthy individual. Any deviations from the two sets of data may be interpreted as indicative of one or more of the disorders. For example, blood samples from type 2 diabetic subjects consistently measured platelet adhesion number of 1.31 fold to 1.72 fold and adhesion rate constants of 1.12 fold to 1.33 fold that of samples from control subjects. Furthermore, the same analysis may furthermore be applied to monitor treatments and provide more timely assay and test results for patients undergoing treatments with pharmaceutical agents including, but not limited to, Coumadin, Warfarin, Plavix and other blood thinners.

Accordingly, in another aspect, the present invention provides an apparatus for the monitoring or assay of blood platelet adhesion or aggregation, the apparatus including a sample flow chamber, a blood flow inlet providing fluid communication into the flow chamber and a blood flow outlet providing fluid communication from said flow chamber, said flow chamber being defined in part by a plurality of sidewalls, and at least one optically transparent wall portion, the at least one said optically transparent wall portion having a chemically inert substrate having a polydimethylsiloxane PDMS compound coated thereon.

### BRIEF DESCRIPTION OF THE DRAWINGS

Reference may now be had to the following detailed description, taken together with the accompanying drawings in which:

FIG. 1A shows a schematic top view of a flow cell having a parallel plate flow chamber in accordance with a preferred embodiment of the invention.

FIG. 1B shows a schematic front view of the parallel plate flow chamber of FIG. 1A.

FIG. 2 illustrates a chemical reaction pathway for cross-linking polydimethylsiloxane (PDMS) to fibrinogen or collagen in accordance with a preferred method.

FIG. 3A illustrates schematically the sample fluid flow path through the parallel plate flow chamber of FIG. 1.

FIG. 3B illustrates velocity profiles at sampling locations across the flow chamber of FIG. 1.

FIG. 4A is a fluorescence image of a selected sampling location after protein bed labelled with fluorescein isothiocyanate (FITC) has been covalently immobilized thereon.

FIG. 4B is a fluorescence image of the selected sampling location after the protein bed labelled with FITC has been covalently immobilized thereon and incubated with Sodium dodecyl sulfate (SDS) for 3 hours and washed with phosphate buffered saline (PBS).

FIG. 4C is a fluorescence image of the selected sampling location after the protein bed labelled with FITC has been non-covalently adsorbed thereon.

FIG. 4D is a fluorescence image of the selected sampling location after the protein bed labelled with FITC has been non-covalently adsorbed thereon and incubated with SDS for 3 hours and washed with PBS.

FIG. 5 is a raw fluorescence image data table of the sampling location showing in rows fluorescence images taken at the start of each kinetic analysis (0 second), then at regular intervals of 40 incremental seconds until 200 seconds after the start.

FIGS. 6A to D show the relationship of platelets adhered over time.

### DETAILED DESCRIPTION OF THE PREFERRED EMBODIMENT

### Parallel Plate Flow Chamber 20

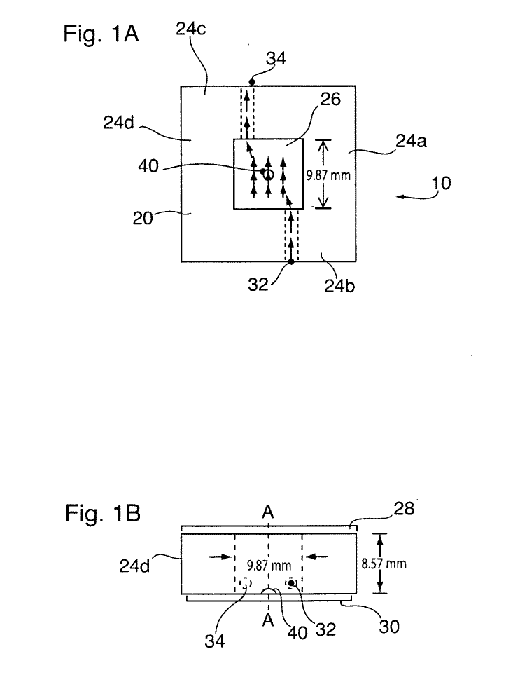

Reference is made to FIGS. 1A and 1B which illustrates a flow cell 10 having parallel plate flow chamber 20 in accordance with a preferred embodiment. The flow chamber 20 is defined radially by a number peripherally extending polytetrafluoroethylene sidewall 24, and is sealed at each of its ends by optically transparent glass cover plates 28, 30. As shown best in FIG. 1A, a blood flow inlet 32 and fluid flow outlet 34 extend through opposing sidewalls 24b, 24c. A chemically immobilized collagen or fibrinogen bed 40 is adhered to the glass cover plate 30 along an axial central (A₁-A₁) portion of the flow chamber 20. As will be described, the parallel plate flow chamber 20 is used for analysis of platelet binding and aggregation to the deposited collagen or fibrinogen bed 40 to assist in the monitoring, diagnosis or treatment of blood or circulatory disorders. In a most preferred use, the flow chamber is used to monitor treatment and/or provide more timely blood test results to patients undergoing treatment with Coumadin, Warfarin, Plavix and/or other forms of blood thinners.

The parallel plate flow chamber 20 is constructed to mimic the in vivo conditions that would facilitate kinetic aggregation studies of blood platelets. The chamber 20 is constructed by cutting a 9.87 mm×9.87 mm square hole through an 8.57 mm-thick block of polytetrafluoroethylene (PTFE) block. Two parallel 15 mm-thick glass cover slips 28, 30 are then sealed over the top and bottom surface of the square Teflon block as illustrated in FIG. 1B. The blood flow inlet 32 is formed by drilling an off centre hole through the sidewall 24 to form a 1.57 mm diameter borehole. The blood flow outlet 34 is likewise drilled off centre through the opposing sidewall 24 to form a 1.57 mm diameter borehole.

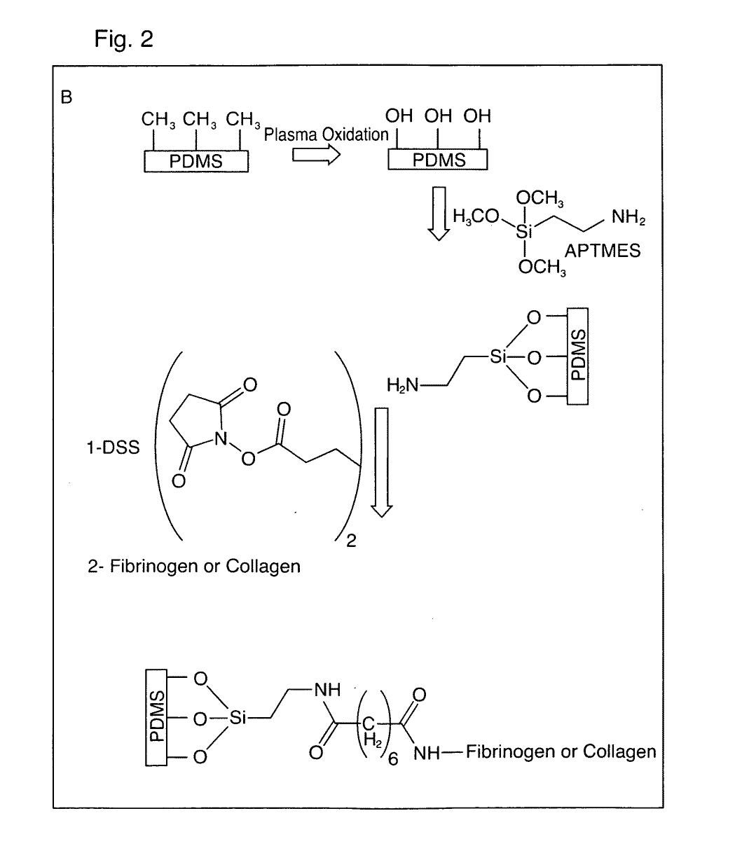

Reference is made to FIG. 2 illustrating the chemical reaction pathway for immobilizing collagen or fibrinogen to form an inlet polydimethylsiloxane (PDMS) bed 40 or the glass cover slip 30. Optionally, a layer of inert PDMS about 0.96 μm thick with 1 mm² surface is poured in the axial center at the bottom of the parallel plate flow chamber 20 at a selected sampling location selected along the axis A₁-A₁, and then plasma activated using a plasma cleaner.

### Plasma Oxidation of PDMS

Preferably, to generate silanol groups on the inert PDMS bed 40 (FIG. 1B), plasma oxidation was carried out using a Plasma cleaner. PDC-32G™ (Harrick Plasma, USA).

### Protein Immobilization Chemistry

After plasma oxidation, 0.2 μl of 2% aminopropyltrimethoxysilane (APTMES) (Sigma Canada) was added on ˜1 mm² surface of PDMS, in the centre of the flow chamber. After 10 minutes, a 0.5 μl portion of 0.5 mM disuccinimidyl suberate (Pierce, USA) solution was added to the APTMES dot in the axial centre of the flow chamber 20. Type 1 fibrinogen form bovine plasma (Sigma) solution with a final concentration of 5 μM was added after 5 seconds. For the experiments with collagen immobilization, 8 μM of collagen type 1 from rat tail (BD Biosciences) was used. The reaction was stopped after 15 minutes by adding Tris buffer pH 8. The immobilization chemistry is shown in FIG. 2.

### SDS Treatment of Protein

Fibrinogen or collagen was fluorescently labelled with FITC and immobilized on PDMS as described above. Fibrinogen-FITC adsorbed on PDMS was used as a control. This was followed by addition of 2% SDS and the samples were incubated for 3 hours. After incubation, PDMS surface was washed with PBS and imaged.

### Subject Selection

Healthy human subjects (n=5), ages 25-40 years were chosen to participate in the study only if they showed no overt symptoms of disease and were taking no medication. Diabetic human subject, ages 25-40 (n=5) on diet therapy alone and achieving stable and satisfactory glycemic control (fasting glycemia and glycosuria variation <15%; post-prandial glycemia variation <25% and HbA1c<7.5%) were chosen for inclusion in the study. None of the patients smoked, had history of alcohol abuse or were taking insulin or any drugs known to lower lipids or interfere with the coagulation and antioxidant systems.

### Blood Collection and Washing of Platelets

Platelets were isolated in a conventional manner, and were labelled with 60 μM BODIPY® FL N-(2-aminoethyl) maleimide (Molecular Probes, Canada). After 30 min incubation, platelets were washed twice with HEPES-ACD™ buffer to remove excess dye and were reintroduced into whole blood.

### Perfusion Studies in Flow Chamber

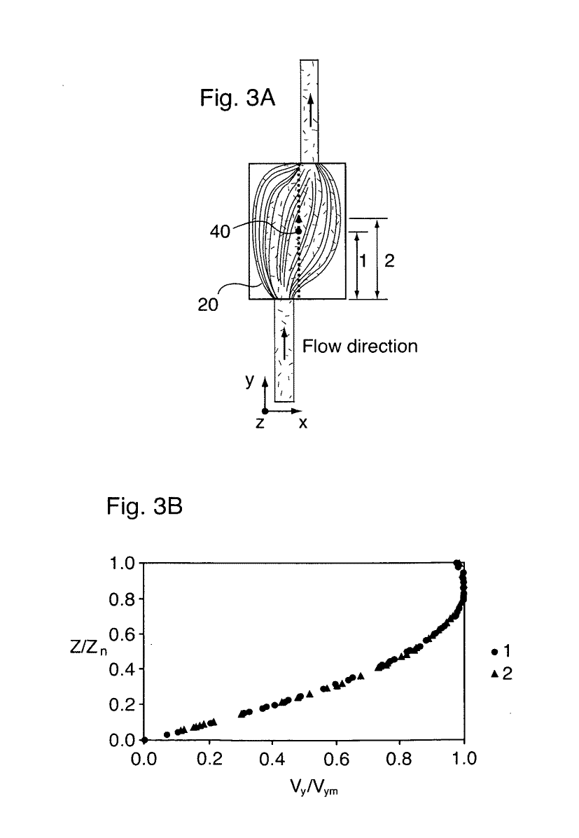

The flow cell 10 circuit was constructed including an in-house designed flow chamber 20 as herebefore described. PTFE sterile tubing (0.031″×0.062″), a medium reservoir, and a peristaltic pump (Reglo Digital™ MS 4/6, model ISM 833, Ismatec), not shown. The offset configuration of the inlet and outlet in the chamber provided optimum flow characteristics desirable for performing and associated studies to be detailed in a subsequent manuscript. FIG. 3A illustrates the flow path lines of the sample through the chamber 20 as simulated by the commercial finite volume solver Fluent™. Flow conditions in the chamber 20 were laminar with Reynolds numbers on the order of 10 based on chamber geometry. The test chamber was open at the top so the flow was that of an open channel. Location 1 and 2 depicted in FIG. 3A represent the spatial regions of interest in this study; these were located 5 mm and 6 mm respectively from the chamber 20 upstream wall 24b. FIG. 3B illustrates the computationally modeled velocity profiles at each location. The profiles show characteristic of the laminar, shearing flow that was desired.

### Results

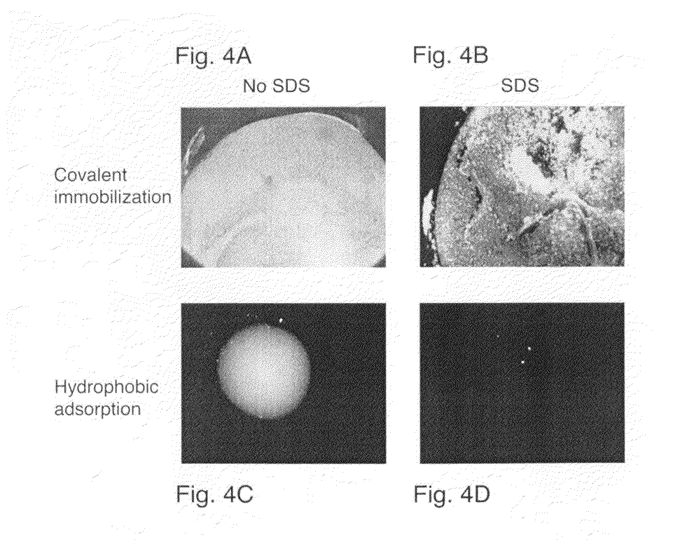

Conventional flow chambers test platelet adhesion and aggregation by coating fibrinogen/collagen on glass slides. In the present invention, an otherwise inert matrix PDMS is plasma activated and used a bifunctional, primary amine-directed crosslinker DSS, to immobilize platelet-binding proteins on the PDMS bed 40. Due to the hydrophobic nature of PDMS, the bed 40 shows high affinity for proteins. In order to show that the adhered proteins (fibrinogen/collagen) in the flow cell 10 are covalently linked and not adsorbed by hydrophobic interactions, the PDMS surface was treated with SDS (2% for 3 h). As can be seen in FIGS. 4A to D, the protein attached to the PDMS-surface via covalent chemistry was not washed away by SDS treatment whereas the control-non covalently attached proteins were totally removed by SDS.

Reference is made to FIGS. 4A, 4B, 4C and 4D which illustrate and confirm covalent immobilization of fibrinogen and collagen proteins to PDMS. FIG. 4A is the fluorescence image of the sampling location after fibrinogen or collagen labelled fluorescently with fluorescein isothiocyanate (FITC) is covalently cross-linked to PDMS. FIG. 4B is the same fluorescence image except the sampling location is incubated 3 hours in sodium dodecyl sulphate (SDS) and washed with phosphate buffered saline (PBS). In comparison, FIG. 4C is the fluorescence image of the sampling location after fibrinogen labelled fluorescently with FITC is non-covalently adsorbed onto PDMS surface. FIG. 4D is the same fluorescence image except the sampling location is incubated 3 hours in SDS and washed with PBS. FIGS. 4B and 4D by comparison illustrates that FITC-labelled proteins are not washed away by the SDS incubation when they are covalently cross-linked to PDMS but not when they are non-covalently adsorbed onto PDMS surface.

### Validation of the Flow Chambers

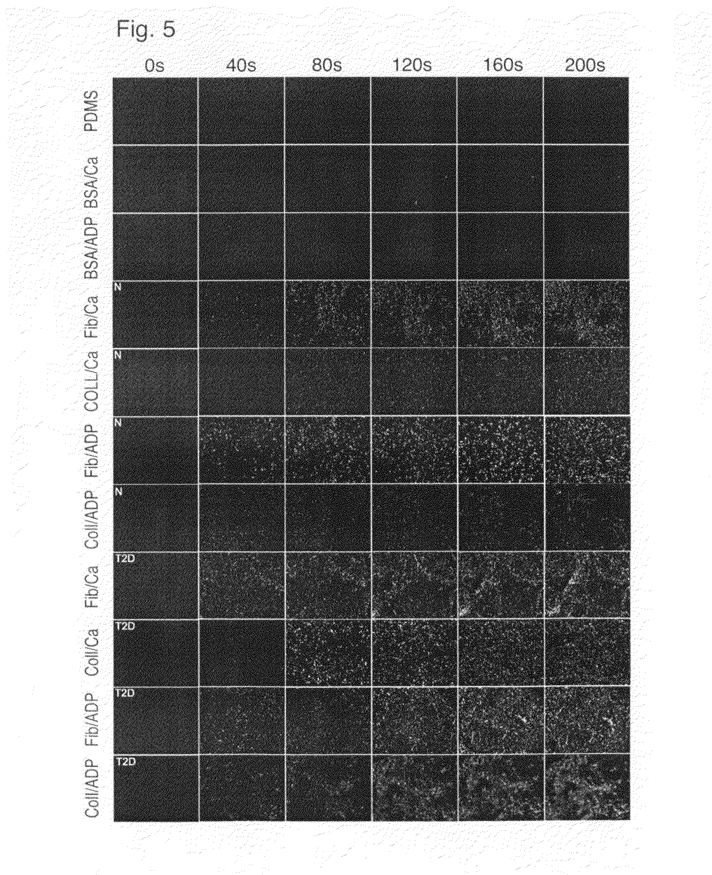

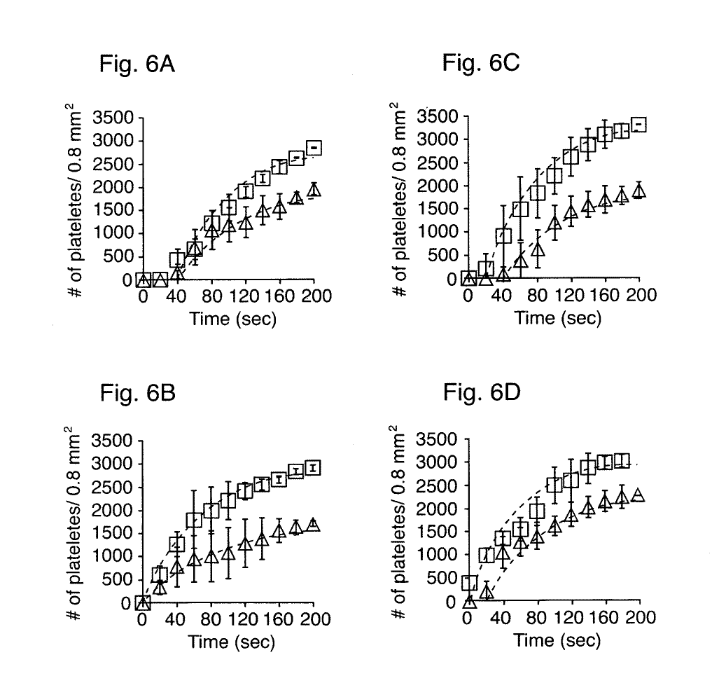

In order to assess the suitability of the flow chamber 20 and the immobilization chemistry for platelet adhesion studies, adhesion to collagen and fibrinogen was tested. Flow chambers 20 were placed onto an inverted fluorescence microscope (Zeiss Axiovert™ 200M). Whole blood containing fluorescently labelled platelets was perfused over the flow chambers 20 containing either PDMS alone or PDMS-immobilized BSA or fibrinogen or collagen. The perfusion rate was 2 ml/min, which generates shear equivalent to that of descending aorta (˜5 dyne/cm²) [9]. The platelets were either activated by the injection of Ca²⁺ (1 mM) or ADP (20 μM) at t=0. Images over the immobilized protein field were captured every 20 s from which the kinetic plots were constructed (FIGS. 6A to 6D). The raw image data, at 40 s intervals, is presented in FIG. 5.

FIG. 5 illustrates a comparison of platelet aggregation in normal and type 2 diabetic subjects. Whole blood containing fluorescently labelled platelets was passed over PDMS alone and PDMS with immobilized BSA, fibrinogen (Fib) or collagen (Coll) using either 1 mM calcium (Ca²⁺) or 20 μM ADP as activators. The images show time dependent increase in platelet adhesion to fibrinogen and collagen, with very little adhesion to the controls (PDMS and BSA). The total number of adherent platelets after 200 sec is greater in the case of type 2 diabetic (T2D) subjects as compared to normal (N) subjects, irrespective of the protein/activator used. All experiments were done at 37° C. Images were captured in 20 sec intervals by Zeiss Axiovert™ 200 microscope with achromat 5× objective (Carl Zeiss), equipped with a Retiga EX cooled monochrome 12 bit camera (Q imaging) and an Xcite series 120 (EXFO, Canada) mercury lamp. Image capture and the quantification of the adhered platelets were facilitated by Northern Eclipse Software (Empix, Canada). FIGS. 6A to D illustrate kinetic plots of platelets adhered to immobilized fibrinogen and collagen over time. Blood reconstituted with fluorescently labelled platelets from normal and diabetic subjects was perfused through the flow chambers containing immobilized fibrinogen and collagen. Graphs 6A and 6B show platelets adhered to collagen and fibrinogen, respectively, over time, when calcium was used as an activator. Graphs 6C and 6D show increasing number of platelets adhered to collagen and fibrinogen respectively, when ADP was used as an activator. The number of platelets adhering over time is greater in diabetic subjects (squares) as compared to normal subjects (triangles). The platelet binding data is extracted from the entire image data set taken at 20 sec intervals. Each point represents the mean±SD from at least t three independent experiments. The solid lines represent the best fit line for the first order treatment of the binding data: Y=Ae^{-kt}.

Platelets are very discriminative with respect to binding sites on fibrinogen or collagen. Since platelets are able to interact with covalently linked fibrinogen/collagen on the applicant's flow cell surface, it strongly suggests that covalent attachment chemistry is not altering collagen/fibrinogen structure.

With the controls, PDMS alone or PDMS-immobilized BSA, there were <50 platelets attached after 200 s of perfusion (FIG. 5). In contrast, when the whole blood was passed over fibrinogen or collagen coated flow cells there occurs a continuous build up in the adhered platelets over the initial density pattern established in the previous time frames indicating that the interaction was irreversible. At the end of the binding experiments ˜10 mL of buffer was pumped through the flow chambers. The number of adhered platelets did not change a further indication of the irreversibility of the interaction with protein matrices covalently attached to PDMS surface.

It is well established that in type 2 diabetic (T2D), the platelets have altered in vitro adhesion and aggregation patterns and are hypersensitive to agonists as compared to the normal platelets. Therefore, an important step in the validation process is to determine whether our flow cells were able to discern functional differences between platelets from normal and T2D subjects. To this end, we compared the adhesion kinetics Ca²⁺ or ADP activated platelets from control and T2D subjects onto immobilized collagen and fibrinogen in the flow chambers. A qualitative examination of the raw data (FIG. 5) revealed that platelets from T2D subjects gave rise to more platelet adhesion irrespective of the activator used or the immobilized surface. These observations were elegantly substantiated upon kinetic treatment of the data shown in FIGS. 6A to 6B and Tables 1 and 2.

In particular, Table 1 shows the total number of normal and type 2 diabetic (T2D) platelets±SD attached to fibrinogen (Fib) and collagen (Coll) after 200 sec, with calcium (Ca²) and ADP as activators.

Table 2 shows the first order rate constants were calculated from the kinetic plots of the platelet adhesion data. The T2D platelets have higher rate constants as compared to the normal (*P<0.05).

TABLE 1
Total number of platelets attached

| | Normal | T2D | Ratio (T2D:N) |
|---|---|---|---|
| Fib/Ca²⁺ | 1920 ± 222 | 3330 ± 150 | 1.73 |
| Coll/Ca²⁺ | 1978 ± 122 | 2859 ± 100 | 1.44 |
| Fib/ADP | 2300 ± 110 | 3024 ± 150 | 1.31 |
| Coll/ADP | 1727 ± 95 | 2904 ± 57 | 1.68 |

TABLE 2
First order rate constants (sec⁻¹)

| | Normal | T2D | Ratio (T2D:N) |
|---|---|---|---|
| Fib/Ca²⁺ | 0.015 ± 0.0005 | 0.0183 ± 0.005* | 1.22 |
| Coll/Ca²⁺ | 0.014 ± 0.0026 | 0.018 ± 0.0011* | 1.28 |
| Fib/ADP | 0.016 ± 0.0011 | 0.018 ± 0.0026 | 1.12 |
| Coll/ADP | 0.012 ± 0.0021 | 0.016 ± 0.0011* | 1.33 |

The total number of platelets bound to the protein surfaces are summarized in Table 1. In general, the maximum number of platelets bound at saturation (˜200 s) was larger for platelets from T2D subjects in comparison to those from the normal subjects, under all conditions examined. The largest ratio between T2D to normal of ˜1.73 was obtained with Ca²⁺-activated platelets on a fibrinogen surface. The smallest ratio of ˜1.31 was obtained with ADP-activated platelets on a collagen surface.

One observation from the kinetic plots of the data was that when platelets were activated with Ca²⁺, with either on the collagen or the fibrinogen surface, there was a 20 s to 40 s lag prior to the initiation of the platelet binding irrespective of the platelet source (T2D or normal) (FIG. 5). In contrast, this lag was absent in the ADP-activated platelets (FIG. 5).

The rate constants extracted from first order kinetic treatment of the data (Table 2) revealed that the binding rate constants for platelets from normal subjects were in general independent of the activator used or the protein immobilized since the differences between the rates (0.014±0.002 sec⁻¹) were not statistically significant. However, when the rate constants of the various activator/protein surface combinations from T2D were compared to normal, in all cases the T2D gave rise to 1.12-fold to 1.33-fold larger and statistically significant, platelet-binding rate constants (0.018±0.002 sec⁻¹).

Although the disclosure describes and illustrates various preferred embodiments, the invention is not so limited. Many variations and modifications will now occur to those skilled in the art. For a definition of the invention, reference may be had to the appended claims.

### REFERENCES

The following publications describe various process and apparatus as related to aspects of the invention heretofore described, and the disclosure of which are hereby incorporated herein by reference.
[1] J. A. Remijn, Y. Wu, E. H. Jening a, M. J. W. Jsseldijk, G. V. Willigen, P. G. Groot, J. J. Sixma, A. T. Nurden, P. Nurden, Thrombosis and vascular Biology 22, 686 (2002).
[2] N. A. Turner, J. L. Moake, L. V. McIntire, Blood 98, 3340 (2001).
[3] I. Goncalves, S. C. Hughan, S. M. Schoenwaelder, C. L. Yap, Y. Yuan, S. P. Jackson, J. Biol Chem 278, 34812 (2003).
[4] K. Miyaki, H. L. Zeng, T. Nakagama, K. Uchiyama, J Chromatogr A 1166, 201 (2007).
[5] S. Miersch, I. Sliskovic, A. Raturi, B. Mutus, Free Radic Biol Med 42, 270 (2007).
[6] H. Chen, L. Wang, Y. Zhang, D. Li, W. G. McClung, M. A. Brook, H. Sheardown, J. L. Brash, Macromol Biosci 8, 863 (2008).
[7] K. B. Neeves and S. L. Diamond, Lab Chip 8, 701 (2008).
[8] E. Gutierrez, B. G. Petrich, S. J. Shattil, M. H. Ginsberg, A. Groisman, Lab Chip 8, 1486 (2008).
[9] T. G. Papaioannou, E. N. Karatzis, M. Vavuranakis, J. P. Lekakis, C. Stefanadis, Int J Cardiol 113, 12 (2006).
[10] A. B. Glassman, Ann Clin Lab Sci 23, 47 (1993).
[11] S. Dittmar, R. Polanowska-Grabowska, A. R. Gear, Thromb Res 74, 273 (1994).
[12] M. El Haouari and J. A. Rosado, Blood Cells Mol Dis 41, 119 (2008).
[13] A. I. Vinik, T. Erbas, T. S. Park, R. Nolan, G. L. Pittenger, Diabetes Care 24, 1476 (2001).

We claim:

1. An apparatus for the monitoring or assay of blood platelet adhesion or aggregation, the apparatus including a sample flow chamber, a blood flow inlet providing fluid communication into the flow chamber and a blood flow outlet providing fluid communication from said flow chamber,
said flow chamber being defined in part by a plurality of sidewalls, and at least one optically transparent wall portion,
the at least one said optically transparent wall portion having a chemically inert substrate having a polydimethylsiloxane PDMS compound coated thereon.

2. The apparatus of claim 1 wherein said monitoring or assaying of blood platelet adhesion comprises empirically measuring blood platelets adhered to the chemically inert substrate within the flow chamber as a measure of blood platelet immobilization.

3. The apparatus of claim 1 wherein said PDMS compound comprises a substantially inert plasma oxidized compound.

4. The apparatus of claim 3 wherein said transparent wall portion comprises a first chamber end wall, said flow chamber is defined in part by a second optically transparent end wall spaced from the first chamber end wall, at least one of the end walls being sized for optically coupling to an inverted optical microscope.

5. The apparatus as claimed in claim 4 wherein the chamber sidewalls comprise PTFE or a PTFE substrate.

6. The apparatus as claimed in claim 5 further including a fluid pump assembly operable to effect a blood sample fluid flow from the blood inlet across said flow chamber and outwardly from said flow chamber via said flow outlet at a rate of 0.5 to 10 ml/per minute and preferably about 2 ml/min.

7. The apparatus as claimed in claim 1 wherein said blood inlet is provided through a first one of said sidewalls, and said flow outlet is provided through a second other opposite sidewall, the blood inlet and outlet being offset with respect to each other to define a non-linear fluid flow path therebetween.

8. The apparatus as claimed in claim 6 wherein the flow inlet and the flow outlet have a spacing selected to provide a laminar, shearing flow therebetween.

9. The apparatus as claimed in claim 8 wherein said flow chamber has a volume selected at between about 0.025 cm³ and 4 cm³, and preferably about 1.0 cm³.

10. Use of the apparatus of claim 3 to optically monitor the therapeutic benefit of a selected hematological treatment agent on a blood platelet function in a blood sample extracted from a patient, said use comprising the steps of;
passing a volume of said blood sample through said flow chamber for a selected period of time; and
measuring the platelet adhesion adhered to the inert oxidized compound within the chamber.

11. Use as claimed in claim 9 wherein said hematological treatment agent comprises a medicament selected from the group consisting of Coumadin, Warfarin, Plavix and an other blood thinner.

12. Use as claimed in claim 11 wherein said step of measuring comprises optically measuring said adhered platelets using an optical microscope.

13. Use as claimed in claim 10 wherein said blood sample comprises a whole blood sample from a patient suffering from a malady selected from the group consisting of Type 2 diabetes, hemophilia, percutaneous coronary intervention, coronary artery disease and coronary stent implantation.

14. A method of making the apparatus of claim 1 comprising the steps of,
applying the PDMS compound coating to at least part of the optically transparent wall portion, and
oxidizing the PDMS compound using a plasma cleaner.

15. The method of claim 14 comprising the further step of applying a homobifunctional cross-linker to the PDMS compound to immobilize platelet binding proteins to less than about 10%, and preferably less that about 2% of a surface area of the transparent wall portion.

16. The method of claim 15 wherein said surface area is located generally at a central axial region of the flow chamber.

17. The method of claim 14 wherein said step of applying the cross-linker comprises adding 1 to 5% by weight, and preferably about 2% by weight aminopropyltrimethoxysilane to a 0.5 to 3 mm diameter section of said transparent wall portion,
applying 0.2 to 1 nM, and preferably 0.5 mM disuccinimidyl substrate solution to said section, and
applying to the section a solution selected from a collagen type 1 solution and a fibrinogen solution prior to buffering.
